(19) United States
(12) Patent Application Publication
(10) Pub. No.: US 2023/0215517 A1
(43) Pub. Date: Jul. 6, 2023

(54) Methods Of Cross Correlation Of Biofield Scans To Enome Database, Genome Database, Blood Test, And Phenotype Data

(71) Applicant: IOLERA HOLDINGS, LLC, Las Vegas, NV (US)

(72) Inventors: Huan Truong, Chandler, AZ (US); Bradley Eckert, Chandler, AZ (US); Bryon Eckert, Chandler, AZ (US)

(21) Appl. No.: 18/115,470

(22) Filed: Feb. 28, 2023

Related U.S. Application Data
(63) Continuation of application No. 16/937,577, filed on Jul. 23, 2020, now abandoned.
Application No. 16/937,577 is a continuation of application No. 15/772,318, filed on Apr. 30, 2018, now abandoned.
(60) Provisional application No. 62/247,265, filed on Oct. 28, 2015.

(51) Int. Cl. G16B 50/10; G16B 20/00; G16H 10/40

(52) U.S. Cl. CPC G16B 50/10; G16B 20/00; G16H 10/40

(57) ABSTRACT

Systems and methods are provided for identifying characteristics of a subject using a biofield scan obtained from the subject. An embodiment can include a method for cross-correlating biofield scans to an enome database, and/or a genome database. A phenotype history and a biofield scan can be created from a user. A user's biofield scan can be created from measured amplitude and frequency. A database is created from a user's phenotype history, and biofield scan. The user's phenotype history and biofield scans are then correlated with known physical and biochemical characteristics. A biofield signature is created and compared to the user's phenotype history, and biofield scan.

### CROSS-REFERENCE TO RELATED APPLICATIONS

This application is a continuation claiming the benefit of priority from U.S. patent application Ser. No. 15/772,318, filed under the same title on Apr. 30, 2018, which is the U.S. National Stage entry of International Application No. PCT/US2016/069011, filed Dec. 28, 2016, which claims the benefit of priority from U.S. Prov. Pat. App. Ser. No. 62/247,265, having the same title, filed on Oct. 28, 2015, all of which applications are incorporated fully herein by reference.

### FIELD OF INVENTION

The present specification relates to methods of cross correlating biofield scans to an enome database, and genome database, with blood tests, and/or phenotype data. More specifically, the present specification relates to a method that correlates biofield scans and phenotypes with existing genome data, and current medical testing, such as, for example, blood tests. Embodiments are not restricted to human biofield and human phenotype correlation, and could be used to correlate other living organisms into an enome or genome database.

Additional features and advantages of the present invention will become apparent to those skilled in the art upon consideration of the following detailed description of the illustrative embodiment exemplifying the best mode of carrying out the invention as presently perceived.

### BACKGROUND

Every living organism has its own biofield. A biofield is a field of energy and information that surrounds every living organism. The energy and information an organism emits is typically at a very low level that requires very sensitive sensors, and equipment that can filter out the surrounding noise. For centuries alternative medicinal practices have used biofield by experienced healers to assist in the analysis of individual state of health. However, currently the missing elements of using biofields in an individual's state of health is the ability to scientifically measure and quantify the biofield, and then correlate the measurements taken to actual health conditions of an organism. U.S. Pat. No. 8,295,903 allows for the ability to take biofield measurements and then quantify the information into a useable database.

### SUMMARY

Aspects disclosed herein comprise a method for correlating multiple results from biofield sensors with the phenotype and disease history of a living organism, such as, for example, humans, mammals, reptiles, or any other living organism. Biofield data can include amplitude peak detections in the extremely high frequency (“EHF”), electromagnetic signals, radio frequency signals, electrical signals, or the like. A biofield sensor, such as, for example, an electron tunneling putative energy analyzer, electron avalanche putative field analyzer, or any other type of sensor that can detect an organism's biofield, can measure biofield data.

In embodiments phenotype data can be collected, which can include both physical and biochemical characteristics of an organism. Phenotype history can be created by the data collected from both physical and biochemical characteristics of an organism, as determined by the interaction of its genetic constitution and environment. Phenotype data can be comprised of a human's medical history such as, for example, history of past illness, hospitalizations, surgeries, immunizations, allergies, personal habits, occupational history, family history, medications, psychiatric history, or the like. Phenotype and biofield data can be stored in a database, and over time the database can allow for individuals to analyze and predict patterns from the data collected.

In embodiments a biofield and phenotype database can identify correlations between biofield scans and existing phenotype data across all organisms, and users. By creating a correlation between biofield scans and phenotype data analysis, the data can continue to improve and become more precise. In embodiments when there is a plurality of biofield scans and a plurality of linked phenotypes in the database, processing within the database can continuously run to improve the quality and precision of the correlations between phenotypes and biofield scans.

In embodiments a relationship between new biofield scans with existing biofield scans database and existing phenotype database, across all subjects and sub-groups of subjects can be created. For example, a new scan of a new subject can be compared against all data in one or more select databases to look for possible health clues in existing phenotype and scan data. This scan can be performed with or without the phenotype of the new scan.

In embodiments a relationship between new and old biofield scans from a single subject with correlations to changes in current health can be created. For instance, comparing a 6 month old scan with a new scan to look for changes in health/wellness. For instance, comparing a scan prior to lunch and after lunch to determine the effects of a specific food on the wellness of an individual. This time dependent scan can be done with or without the phenotype of the individual, but is best performed with a phenotype of record and an update of the phenotype data.

In embodiments a relationship between “enome” and genome correlations can be created. This is to allow the extremely rich existing genome data bases to be used for possible clues into the state of health of an individual based on a bioscan. The goal is to allow correlations in bioscans and genome to provide possible links that assist in understanding the implications of a bioscan.

In embodiments a relationship between enome and blood test correlations can be created. For example, if a strong correlation between bioscan results and blood sugar level can be found then a bioscan could be used as one indicator that a blood sugar test is urgently needed and or a diet change/insulin injection is required.

### BRIEF DESCRIPTION OF DRAWINGS

The detailed description of the drawings particularly refers to the accompanying figures in which:

FIG. 1 is a diagram showing a correlation of phenotype items to biofield scans;

FIG. 2 is a diagram showing a correlation of biofield scan patterns to phenotype data;

FIG. 3 is a diagram showing differential biofield scans per user over time;

FIG. 4 is a diagram showing enome tags and genetic markers for fast data comparisons;

FIG. 5 is a flowchart illustrating an exemplary method of biofield scan analysis by deoxyribonucleic acid (“DNA”) markers;

FIG. 6 is a flowchart illustrating an exemplary method of correlating biofield scans to blood test results;

FIG. 7 is a flowchart illustrating an exemplary method of selecting an appropriate blood or clinical test to compare to a biofield scan; and

FIG. 8 is a diagram showing a sample enome life cycle.

### DETAILED DESCRIPTION

The embodiments described herein are not intended to be exhaustive or to limit the invention to precise forms disclosed. Rather, the embodiments selected for description have been chosen to enable one skilled in the art to practice the invention. The described embodiments lends themselves to many variants of systems and methods for interpreting “scans,” or detected and recorded information, of a subject's biofield, and correlating the biofield scans phenotype and other genomic information. Various embodiments as described in the present disclosure may create or use “enome” information, including recorded and/or processed data, stored data elements and data records, files, databases, etc., that is relevant to a subject or group of subjects. Like a genome constitutes some or all of the characteristics of the genetic material (e.g., coding and/or noncoding regions of DNA) of an organism, including phenotypical and various types of genetic relationships to other organisms, an “enome” as used herein constitutes some or all of the characteristics, including but not limited to visible, determinable, and relational characteristics, of an organism's biofield.

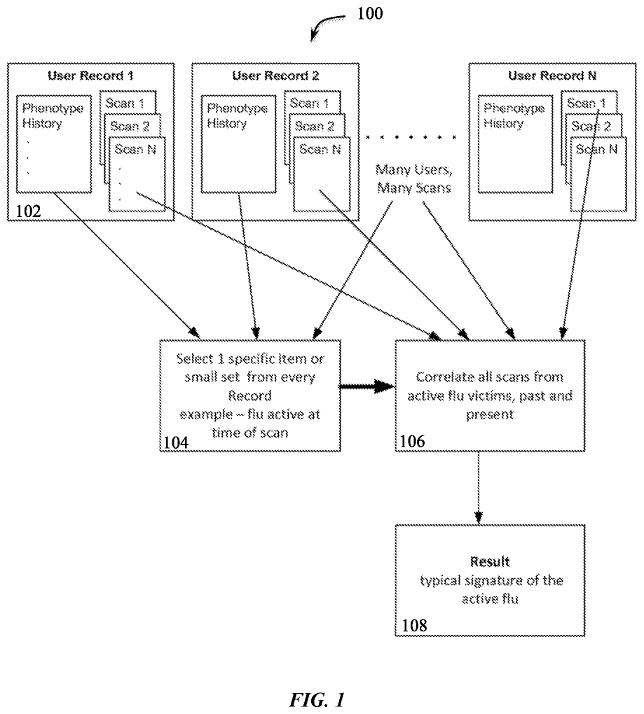

Referring initially to FIG. 1, an illustrative method 100 may be used for correlating user specific phenotypes and then matching the characteristics in enome biofield scans. At step 102, a system performing the method may provide phenotype history of a plurality of users for a specific data point or multiple data points. Step 104 includes selecting a specific data point or multiple data points from the collected user's phenotype history and then classifying a specific data point or multiple data points into determinable groups, such as, for example, active flu symptoms, inactive flu symptoms, active cold symptoms, inactive cold symptoms, active cancer symptoms, inactive cancer symptoms, or any other type of virus or disease that can affect a user. At step 106, signature characteristics of active flu symptoms, active cold symptoms, active cancer symptoms, or any other type of active virus or disease (hereinafter “active condition”) can be correlated and determined by the specific data point or multiple data points from past and present symptoms of the users. At step 108, a typical signature pattern can then be identified in the biofield scans of user record 1, and checked and refined, using the correlated data point(s), against other user records to best differentiate a user's active symptoms, producing a typical biofield signature for the specific data point (e.g., a user's symptom) or multiple data points. For example, the method 100 may produce a biofield signature for an active flu using all of the data points from the phenotype histories that are relevant to the active flu.

In certain embodiments data can be extracted from a fast Fourier transform (“FFT”), which can consist of a list of amplitude peaks at corresponding frequencies. Amplitude peak data can be sorted by frequency and it can be a primary source for stored bioscan information. In certain embodiments bioscan data pertaining to a user can be stored in the associated user record using 1-byte, 2-byte, 3-byte, 4-byte, 5-byte, 6-byte, 7-byte, 8-byte, 9-byte, 10-byte, 11-byte, 12-byte, etc., record structure.

In embodiments it is not necessary to save every point of FFT output, just amplitude peaks and/or the corresponding frequencies where amplitude peaks occur. In certain embodiments there could be 1 to 50 million FFT points in a scan, but the data saved may be limited to a few thousand peaks. In embodiments, the peaks may be important for determining the input searches. The number of peak frequencies, the range of frequencies, and the resolution is expected to change over time as the instruments improve in speed, sensitivity and range. Data compression may be routinely used; a data specific compression technique may be used.

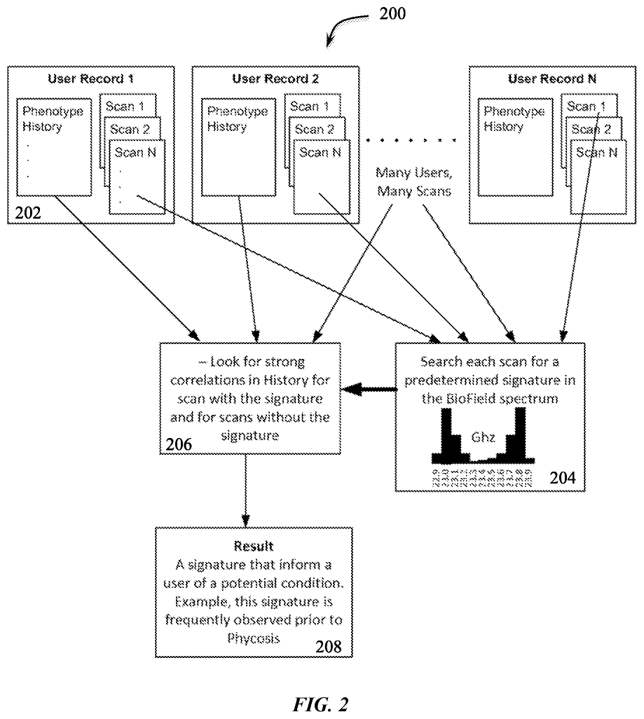

Referring to FIG. 2, an illustrative method 200 may be used for analyzing an enome database and then matching patterns such as, for example, a user's past or present medical history from a plurality of scans. At step 202 a plurality of scans can be taken from a user from a user's phenotype history, bioscan history, or both. A unique signature can be found from the user's phenotype history and/or bioscan history, and then the unique signature can be sorted through to find a common signature which can be common to some users, but not all users. At 204, each scan can be searched for a predetermined signature in a biofield spectrum, such as, for example, 20 GHz, 21, GHz, 22 GHz, 23 GHz, 24 GHz, or the like. At 206, a correlation between the user's phenotype and/or bioscan history, and an active condition's signature can be determined, and separated from those scans without an active condition signature. At 208, a signature can be created that can inform a user of a potential active condition, such as a signature that can be frequently observed prior to diagnosis.

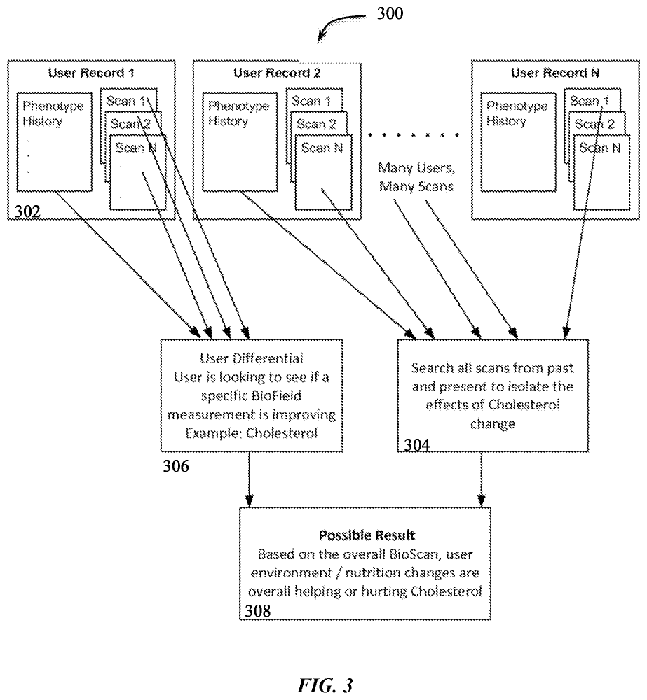

Referring to FIG. 3, an illustrative method 300 may be used for analyzing an enome database over a period time for a single user. At step 302, phenotype and/or bioscan history can be measured over a period of time for a single user. At step 304, a user's phenotype and/or bioscan history can be searched from either past or present, or both past and present scans to determine and isolate the effects of such as, for example, a change in the user's cholesterol, diabetes, blood pressure, or the like. At step 306, a differential measurement can be determined between a user's past specific biofield measurement and a user's present specific biofield measurement. The differential measurement can be used to determine whether the user's cholesterol, diabetes, blood pressure, or the like has changed or improved over a period of time. At 308, based on an overall bioscan and/or phenotype history the system can determine whether a user's environment, nutrition, exercise regime, or the like can be beneficial or a detriment to the user when trying to alter the user's cholesterol, diabetes, blood pressure, or the like.

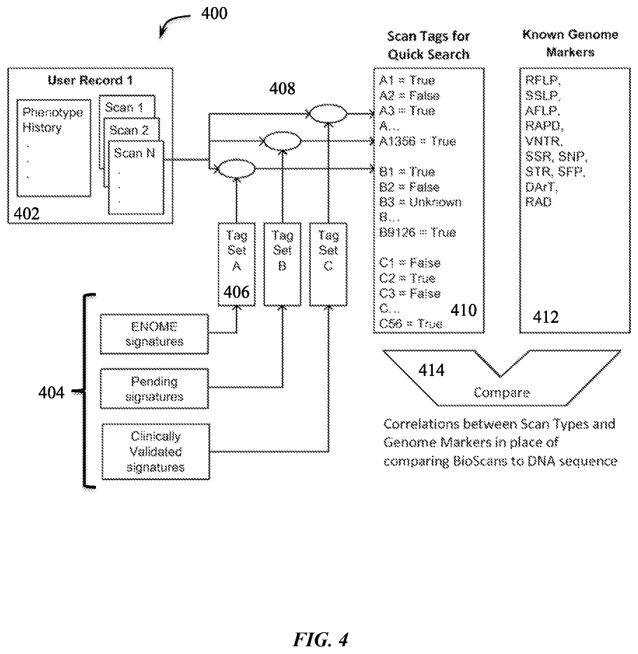

FIG. 4 illustrates an exemplary method 400 to organize (i.e., create and/or modify) and search a correlation database of correlated biofield scans in accordance with the present disclosure. At step 402, a known user record can be stored in such as, for example, one, two, three, four, five, six, seven, eight, nine, or the like databases. At step 404, biofield signatures can be stored in a database and then can be assigned a signature class, such as, for example, enome signatures, clinically validated signatures, and signature not yet determined or identified. At step 406, enome signatures, clinically validated signatures, and signature not yet determined or identified can be assigned a tag. At step 408, using a user's record history and comparing it with the enome signatures, clinically validated signatures, and pending signatures a correlation can be applied to determine whether the signatures match the user's record history. At step 410, a scan can be completed of each signature in each signature database, and the tags can be set to either true or false depending upon the signature and the user record history. At step 414, in certain embodiments known markers 412—such as full genome sequences, genetic markers, or phenotype markers— can be directly compared and correlated to the scan tags. In embodiments, as new signatures are added, only the new and/or altered signatures need to be compared against existing scans to update the tags for each scan.

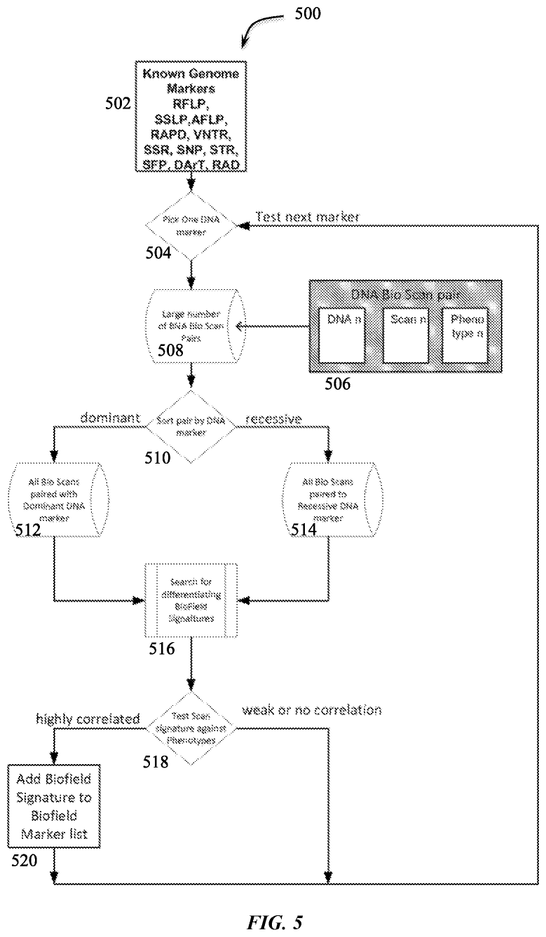

FIG. 5 illustrates an exemplary method 500 to analyze biofield scans by correlation to DNA or other genetic markers. At step 502, known genetic markers such as, for example, restriction fragment length polymorphism (“RFLP”), simple sequence length polymorphism (“SSLP”), amplified fragment length polymorphism (“AFLP”), random amplified polymorphic DNA (“RAPD”), variable number tandem repeat (“VNTR”), simple sequence repeat (“SSR”), single-nucleotide polymorphism (“SNP”), short tandem repeat (“STR”), single feature polymorphism (“SFP”), Diverse Arrays Technology (“DArT”), restriction-site associated DNA (“RAD”), or the like are identified. At step 504, from the known genetic markers one or more than one marker is separated from the other genetic markers. At step 508, known genome and/or DNA markers, and a user's bioscan history 506 (which may be stored in a user record of a database as described above and may contain, for example, one or more DNA bioscan, scan of a user, and phenotype) are sorted and organized into two or more pairs. DNA markers can be selected from markers that may have a known relationship to a genetic makeup of a user.

At step 510, the pairs from step 508 can be sorted by their DNA marker traits, such as, for example, a dominant or recessive trait. At step 512 and 514, the user's bioscan can be paired to its DNA marker and then it can be separated into either a recessive or dominant DNA marker. At step 516, the paired recessive or dominant DNA markers can be scanned for differentiating biofield signatures. At step 518, the test scan of the biofield signatures can be compared against phenotypes to determine whether there can be either a high correlation, weak correlation, or no correlation between the phenotypes and biofield signatures. The bioscans can be searched to find patterns that match in each group and contrast to patterns that may be found in other groups. At step 520, if a correlation can be found in the bioscans the common pattern will be considered as a possible biofield pattern of significance, and if there is a high correlation between the phenotypes and biofield signature, the biofield signature can be added to the biofield marker list.

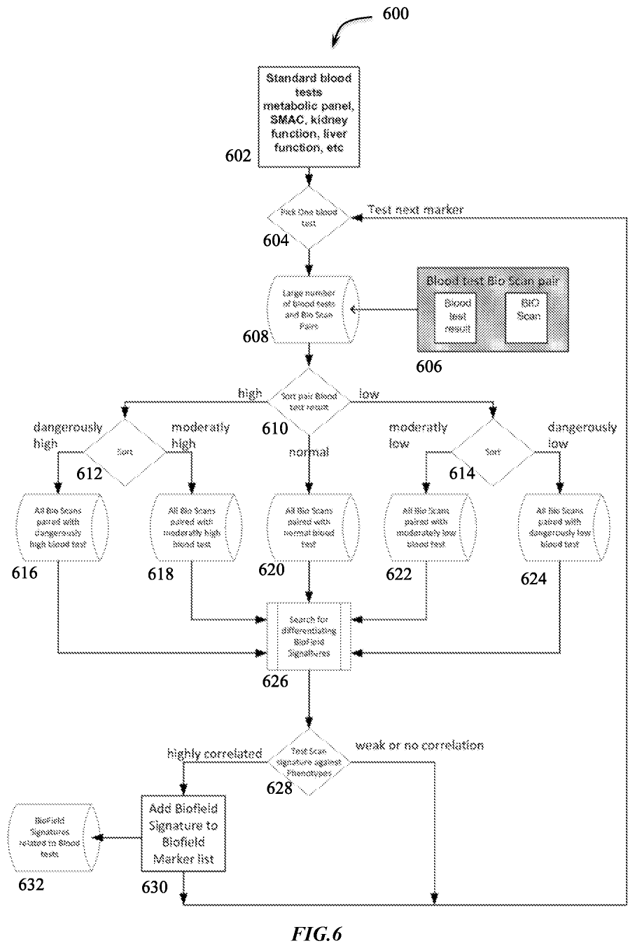

FIG. 6 illustrates an embodiment for a method 600 for analyzing a biofield scan and correlating it to a user's blood test. At step 602, a standard blood test such as, for example, metabolic panel, sequential multiple analysis by computer (“SMAC”), kidney function, liver function, or the like can be identified. At step 604 a standard blood test can be selected. At step 608, a user's blood from the user's bioscan and/or blood test results can be organized and then compared to the standard blood tests. To aid in identifying significant biofield signatures, paired sets 606 of blood tests and bioscans can be analyzed. At step 610, 612, and 614, bioscans are organized into two or more groups based upon the results of the blood test and then can be sorted into high, low, or normal significance. In certain embodiments a blood test can be drawn from the user and/or test subject at the same time the bioscan is taken or after the bioscan is taken. At step 616, 618, 620, and 622 a bioscan can be paired with a blood test and then can be sorted into groups of such as, for example, dangerously high, moderately high, normal, moderately low, and dangerously low.

Once the bioscans are sorted by blood tests results, the bioscans are then searched to find patterns that can match each group and contrast patterns found in other groups. At step 626, if a correlation is found in the bioscan the common pattern can be considered as a possible biofield pattern of significance. In embodiments it can be expected that a noninvasive bioscan can be used as a prescreening to determine what blood tests are likely to be useful. At step 628, the test scan of the biofield signatures can be compared against phenotypes to determine whether there can be either a high correlation, weak correlation, or no correlation between the phenotypes and biofield signatures. The bioscans can be searched to find patterns that match in each group and contrast to patterns that may be found in other groups. At step 630, if a correlation can be found in the bioscans the common pattern will be considered as a possible biofield pattern of significance, and if there is a high correlation between the phenotypes and biofield signature, the biofield signature can be added to the biofield marker list. At step 632, biofield signatures related to blood tests can be determined and correlated. In certain embodiments a bioscan can be used to prescreen what blood tests can be useful to a user. In an exemplary embodiment scanning blood in vitro can create the best correlation between a bioscan and a blood sample.

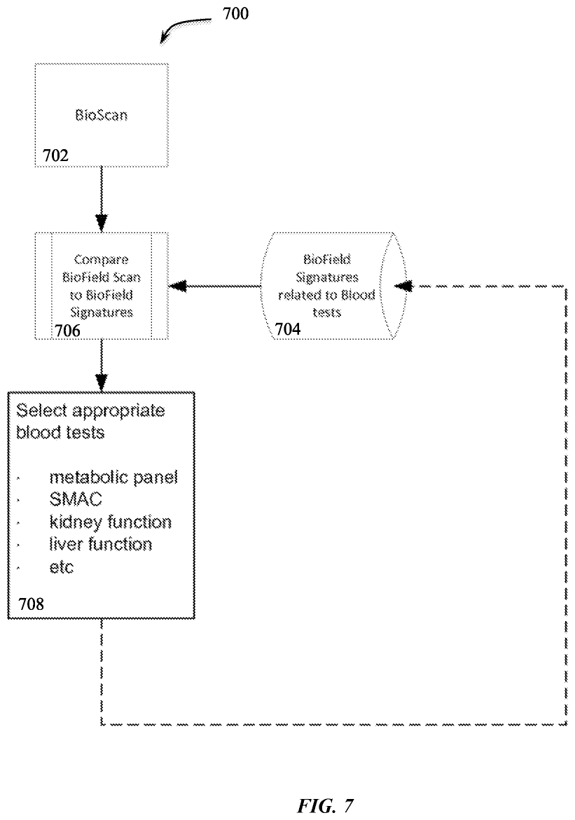

FIG. 7 illustrates a certain embodiment of a method 700 for using bioscans as a prescreening prior to ordering a blood test. At step 702, a bioscan can be used to minimize unnecessary testing and to insure that necessary test is completed. At step 704 and 706, biofield signatures related to blood tests or any other type of testing done on a user in the enome database can be compared to a bioscan. In an exemplary embodiment database built over time can help minimize unnecessary testing and insure that needed testing can be completed. At step 708, a blood test can be selected such as, for example, metabolic panel, SMAC, kidney function, liver function, or the like, and then it can be fed back into a biofield enome database to assist in the selection of an appropriate test for future scans.

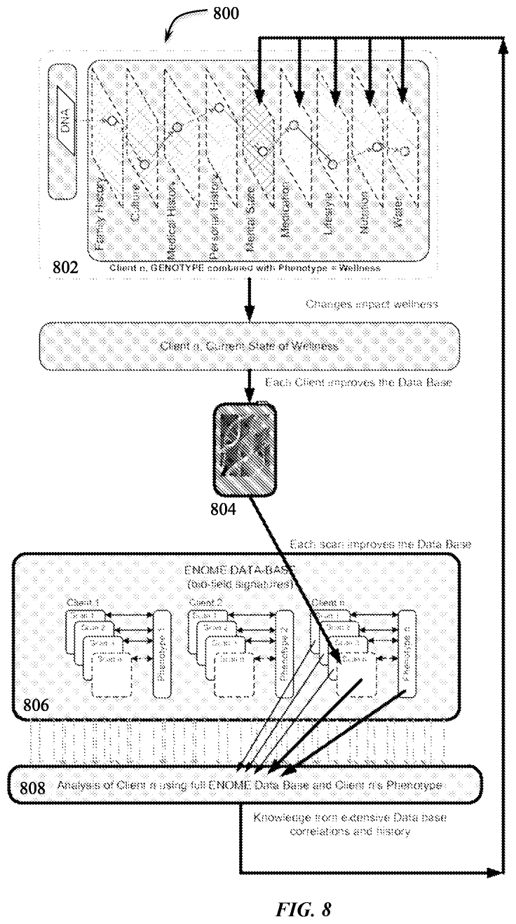

FIG. 8 generally illustrates an enome life cycle 800 and its intended use in an enome database. At step 802 and 804, an exemplary embodiment can have a user with a combined genotype, and phenotype database, which can be an enome database. An enome database can comprise of such as, for example, family history, culture, medical history, personal history, mental state, medication, lifestyle, nutrition, and water, which can create a user's current state of wellness. At step 806, adding and using existing users and their phenotypes, and biofields can continuously expand an enome database to be able to correspond to any state of wellness of a user. Each scan created can continue to fill in the enome database and improve its accuracy over time. At step 808, in embodiments a user can use a fully populated enome database and users phenotypes to create a wellness plan for that user.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

### EXAMPLES

The following non-limiting example is provided for illustrative purposes only in order to facilitate a more complete understanding of representative embodiments. This example should not be construed to limit any of the embodiments described in the present specification including those pertaining to the method of cross correlating biofield scans to an enome database, and genome database, with blood tests, and/or phenotype data.

### Example 1

### Matching a Pattern of Frequency Spikes in a Biofield Signature

When a unique signature is found that is common to some but not all individuals, the biofield signature can be defined. For example, when two strong amplitude spikes are found at frequencies 23.0 GHz, and 23.8 GHz with no amplitude peaks between, this signature of peaks can then be correlated to a phenotype history of all users and the correlations can then be searched. An unexpected relationship between a represented medical condition and a biofield signature can exist. Amplitude spikes are not limited to two or three or four, but can involve thousands if not millions of peaks and valleys to correlate to a user's phenotype and/or biofield history.

We claim:

1. A method of generating a correlation database storing data that correlates biofield characteristics to phenotypes of one or more organisms, the method comprising:
obtaining a plurality of user records each associated with a corresponding subject of a plurality of subjects, each user record comprising:
one or more data points representing a phenotype history of the corresponding subject; and
a first biofield scan comprising biofield data obtained by scanning the corresponding subject's biofield;
correlating the one or more data points of each user record across the plurality of user records to produce a correlated phenotype;
using the correlated phenotype to determine a biofield signature present in the biofield data of the corresponding first biofield scan of each of the plurality of user records; and
producing a record that associates the biofield signature with the correlated phenotype; and
storing the record in the correlation database.

2. The method of claim 1, wherein the biofield data comprises frequency data and amplitude data associated with the frequency data, and wherein using the correlated phenotype to determine the biofield signature comprises identifying a pattern of amplitude peaks at particular frequencies.

3. The method of claim 2, wherein identifying the pattern of amplitude peaks comprises applying a fast Fourier transform to the biofield data of the corresponding first biofield scan of each of the plurality of user records to produce a desired number of the amplitude peaks.

4. The method of claim 1, wherein the corresponding one or more data points of each of the plurality of user records indicate whether the corresponding subject is exhibiting one or more symptoms of an active condition, and wherein producing the record comprises associating the biofield signature with the active condition.

5. The method of claim 1, wherein producing the record comprises assigning a signature class to the biofield signature, the signature class indicating whether the biofield signature is clinically validated.

6. The method of claim 1, wherein producing the record comprises assigning a signature class to the biofield signature, the signature class indicating whether the biofield signature is an enome signature.

7. The method of claim 1, wherein producing the record comprises:
selecting, based on the phenotype history represented by at least one of the plurality of user records, a first scan tag from a plurality of scan tags each correlated to a corresponding marker of a plurality of known markers, the known markers including one or both of a genetic marker and a phenotype marker; and
assigning the first scan tag to the biofield signature.

8. The method of claim 1, further comprising generating a plurality of biofield marker lists each associated with a corresponding genetic marker of a plurality of genetic markers, and each biofield marker list listing biofield signatures stored in the correlation database that have a high correlation with the phenotypes that are related to the corresponding genetic marker.

9. The method of claim 1, further comprising generating a plurality of biofield marker lists each associated with a corresponding blood test of a plurality of blood tests, and each biofield marker list listing biofield signatures stored in the correlation database that have a high correlation with the phenotypes that are related to the corresponding blood test.

10. The method of claim 9, wherein generating the plurality of biofield marker lists comprises:
before producing the correlated phenotype:
obtaining a blood test result obtained by performing a first blood test of the plurality of blood tests on a first subject of the plurality of subjects;
pairing the corresponding first biofield scan of a first user record of the plurality of user records with the blood test result, the first user record being associated with the first subject; and
based on the blood test result, selecting a first group from a plurality of groups, the first group including the plurality of user records; and
after determining the biofield signature:
determining a high correlation between the biofield signature and the phenotypes associated with the first blood test; and
adding the biofield signature to the biofield marker list associated with the first blood test.

11. A method of correlating biofield scans to phenotype data of one or more organisms, the method comprising:
providing a phenotype history of a user;
providing a plurality of biofield scans of said user, wherein said biofield scans are measured in frequency and amplitude;
creating a database with said phenotype history and said biofield scans of said user;
correlating said phenotype and said biofield scan within said database;
creating a biofield signature from said phenotype history, and said biofield scans;
comparing said biofield signature with said phenotype history, and said biofield scan of said user; and
outputting said biofield signature and said phenotype history, and said biofield scan comparison.

12. The method of claim 11, wherein said phenotype history is provided from more than one user.

13. The method of claim 11, wherein said biofield signatures are used to generate biofield tags.

14. The method of claim 13, wherein said biofield tags are compared to said phenotype history and said biofield scans.

15. The method of claim 11, wherein said biofield scans are compared to genetic markers.
